(19) United States
(12) Patent Application Publication
(10) Pub. No.: US 2025/0127543 A1
(43) Pub. Date: Apr. 24, 2025

(54) HANDLE FOR REDUCING LENGTH OF BONE SCREWDRIVER BIT

(71) Applicant: OrthoPediatrics Corp., Warsaw, IN (US)

(72) Inventor: Camden Fox, Fort Wayne, IN (US)

(21) Appl. No.: 18/918,945

(22) Filed: Oct. 17, 2024

Related U.S. Application Data
(60) Provisional application No. 63/545,023, filed on Oct. 20, 2023.

(51) Int. Cl. A61B 17/70; A61B 17/00

(52) U.S. Cl. CPC A61B 17/7082; A61B 2017/0046

(57) ABSTRACT

An apparatus includes a handle, and a retention assembly. The handle extends between a proximal portion and a distal portion and also defines a shaft receiving channel extending from an end of the distal portion into the proximal portion of the handle. The handle also includes a torque generating surface. The shaft receiving channel is dimensioned to receive an elongated navigation unit coupling body of a navigation compatible bone screwdriver bit, thereby reducing its effective length. The torque generating surface transmits torque onto the navigation compatible bone screwdriver bit while the elongated navigation unit coupling body is housed within the shaft receiving channel. The retention assembly inhibits decoupling of the handle and the screwdriver bit while the elongated navigation unit coupling body is housed within the shaft receiving channel.

### BACKGROUND

Spinal fixation systems may be used to surgically fix, adjust, and/or align the spinal column. One type of spinal fixation system employs a spinal rod for supporting the spine and fixing, adjusting, and/or aligning all or portions of the spinal column into a desired orientation. Attachment of the spinal rod to the spinal column has been achieved using a variety of vertebral anchors. Pedicle screws have been used successfully as vertebral anchors. Pedicle screws and connectors in combination with spinal rods can align and correct deformities in the natural spinal alignment as well as repair traumatic injury. In general, a pedicle screw has a head with a receiving opening into which a spinal rod can be secured.

Pedicle screws may be driven into a pedicle of a vertebra of a patient utilizing a suitable bone screwdriver bit attached to a handle or other suitable torque generating device. Bone screwdriver bits are configured to transfer torque from the handle onto the pedicel screw to thereby drive the pedicle screw into the vertebra. In some instances, pedicle screws may be driven into a pedicle of a vertebra with the assistance of a navigation system. Such a navigation system may include a tracking device attached to a bone screwdriver bit at a fixed spatial location, where the navigation system is configured to track the location and spatial orientation of the tracking device, and therefore the bone screwdriver bit, during illustrative use. Navigation systems may utilize the tracked location and orientation of the bone screwdriver bit in order to further assist a surgeon.

While various kinds of handles for bone screwdriver bits have been made and used, it is believed that no one prior to the inventor(s) has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of a navigation compatible bone screwdriver bit decoupled from an illustrative shaft reduction handle;

FIG. 2 depicts an perspective view of the shaft reduction handle of FIG. 1;

FIG. 3 depicts a sectional view of the shaft reduction handle of FIG. 1, taken along line 3-3 of FIG. 1;

FIG. 4 depicts a sectional view of the shaft reduction handle of FIG. 1, taken along line 4-4 of FIG. 1;

FIG. 5A depicts a cross-sectional view of the shaft reduction handle of FIG. 1 detached from the navigation compatible bone screwdriver bit of FIG. 1, with a driver retention assembly of the shaft reduction handle in a locked configuration;

FIG. 5B depicts a cross-sectional view of the shaft reduction handle of FIG. 1 detached from the navigation compatible bone screwdriver bit of FIG. 1, with the driver retention assembly of FIG. 5A in an unlocked configuration;

FIG. 5C depicts a cross-sectional view of the shaft reduction handle of FIG. 1, with the navigation compatible bone screwdriver bit of FIG. 1 initially inserted into a shaft receiving channel of the handle, with the driver retention assembly of FIG. 5A in the unlocked configuration;

FIG. 5D depicts a cross-sectional view of the shaft reduction handle of FIG. 1, with the navigation compatible bone screwdriver bit of FIG. 1 fully inserted into a shaft receiving channel of the handle, with the driver retention assembly of FIG. 5A in the locked configuration; and

FIG. 6 depicts a perspective view of the shaft reduction handle of FIG. 1 coupled with the navigation compatible bone screwdriver bit of FIG. 1, where the screwdriver bit is driving a pedicle screw into a vertebra of a patient.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

### I. Illustrative Shaft Reduction Handle Configured to Couple With Navigation Compatible Bone Screwdriver Bit

As mentioned above, some bone screwdriver bits are configured for use with navigation systems such that a tracking device of a navigation system may selectively attach to the bone screwdriver bit, thereby enabling the navigation system to track the location and orientation of a bone screwdriver bit during illustrative use. In order to accommodate attachment of a tracking device of a navigation system, the length of a navigation compatible bone screwdriver bit may be increased in order to provide the necessary room to couple such a tracking device at a location that would not interfere with the operation of a bone screwdriver bit.

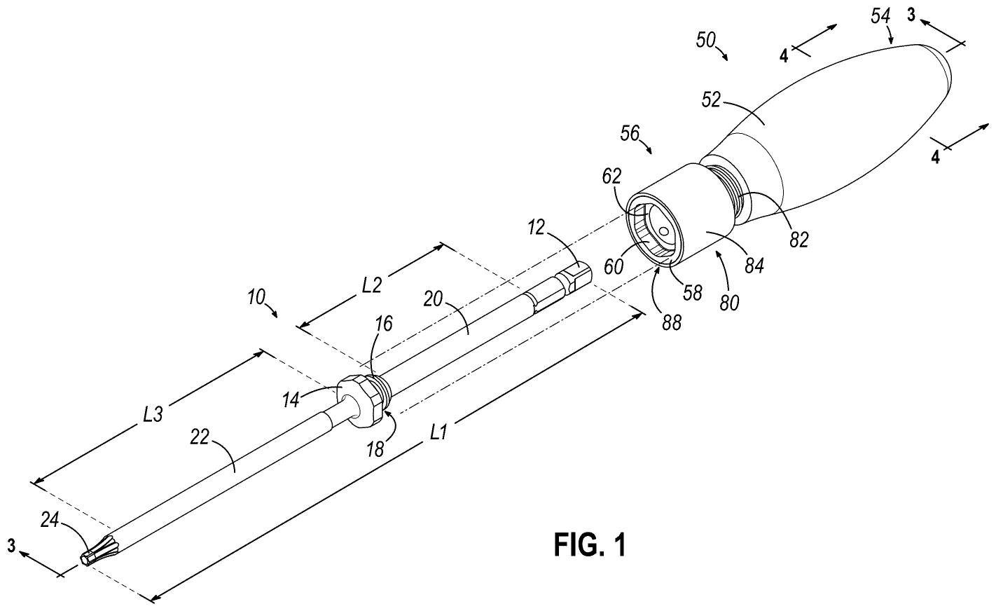

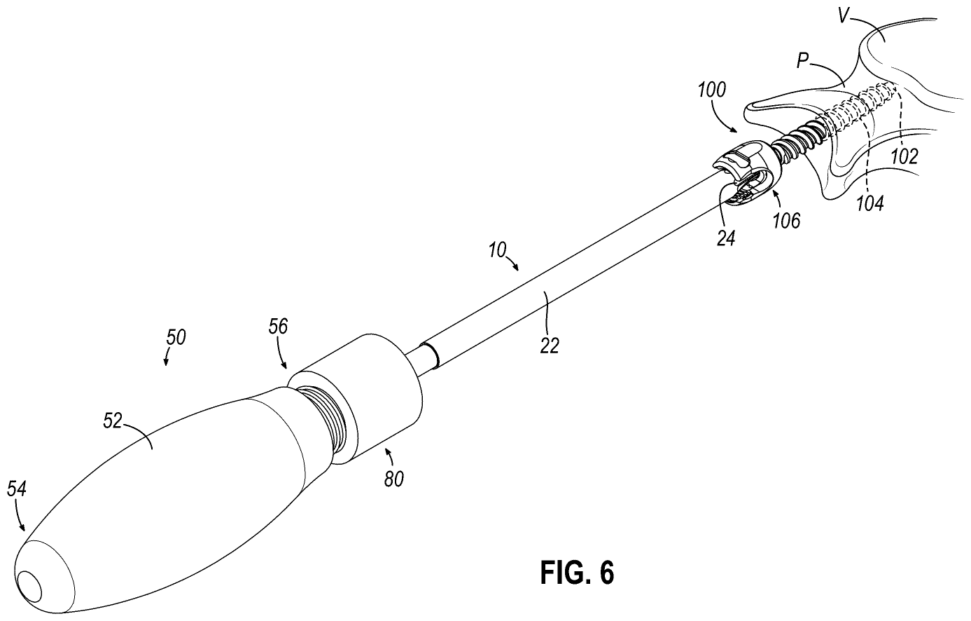

FIG. 1 shows an illustrative navigation compatible bone screwdriver bit (10) having an overall length (L1) that is configured for use with a navigation system. Bone screwdriver bit (10) includes a first torque receiving body (12), a second torque receiving body (14), a collar (16) located adjacent to the second torque receiving body (14), an elongated navigation unit coupling body (20), a distal shaft (22), and a screw bit (24). As shown in FIG. 6, screw bit (24) is configured to selectively engage and apply torque to a pedicle screw (100) (see FIG. 6) (or any other suitable bone screw or anchor) in order to anchor pedicle screw (100) to vertebrae (V) of a patient. Such a pedicle screw (100) includes a tulip head (106) and a shank (102) having a thread (104) helically disposed along a suitable length of shank (102). Shank (102) and thread (104) are configured to be anchored into vertebra (V) of a patient. Pedicel screw (100) may have any suitable features, as would be apparent to one skilled in the art in view of the teachings herein, configured to be driven by screw bit (24) in order to drive/anchor shank (102) and thread (104) into the pedicel (P) of a vertebra (V).

Elongated navigation unit coupling body (20) is configured to couple to a tracking device of a navigation system. Therefore, elongated navigation unit coupling body (20) has a suitable length (L2) spaced away from screw bit (24) via the length (L3) of distal shaft (22) such that an attached tracking device does not interfere with the operation of screw bit (24). When utilizing a tracking device, first torque receiving body (12) may be attached to a torque generating device (e.g., a handle) such that elongated navigation unit coupling body (20) extends distally from such a torque generating device. The portion of bone screwdriver bit (10) extending distally from a handle (or other suitable torque generating device) may be considered the effective length of the bone screwdriver bit (10).

However, in some instances, it may be desirable to use navigation compatible bone screwdriver bit (10) (or any other suitable navigation compatible bone screwdriver bit) without the assistance of a navigation system. In instances where navigation units are not used in conjunction with bone screwdriver bit (10), elongated navigation unit coupling body (20) is not attached to a tracking device. In such instances, having the length (L2) of navigation unit coupling body (20) extend distally from a handle (or other torque generating device) may not be required, or even desired.

Therefore, it may be desirable to have a handle that is configured to be used with navigation compatible bone screwdriver bit (10) when not used in conjunction with a navigation system; and is also configured to reduce the effective length of bone screwdriver bit (10). Reducing the effective length of bone screwdriver bit (10) may provide various advantages that will be apparent to one skilled in the art in view of the teachings herein. As one example, reducing the effective length may provide greater control of bone screwdriver bit (10) during illustrative use.

FIGS. 1-4 show an illustrative handle (50) that may be used in conjunction with navigation compatible bone screwdriver bit (10). Handle (50) includes a handle body (52) extending between a proximal end (54) and a distal end (56), and a driver retention assembly (80). As will be described in greater detail below, illustrative handle (50) is configured to: receive bone screwdriver bit (10) to reduce its effective length; suitably engage torque receiving bodies (12, 14) to generate suitable torque at screw bit (24) to thereby anchor a pedicle screw (100) to a vertebrae (V) of a patient; and retain bone screwdriver bit (10) to inhibit inadvertent decoupling of bone screwdriver bit (10) from handle (50).

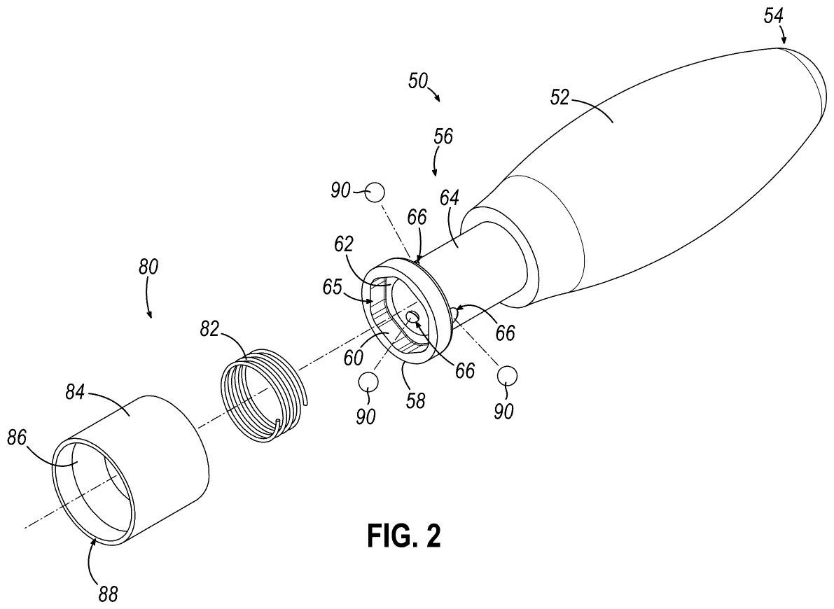

Turning to FIG. 2, distal end (56) of handle body (52) includes a distal torque transfer body (58) and collar (64) extending proximally from distal torque transfer body (58). Distal torque transfer body (58) includes a plurality of torque transfer surfaces in the form of flat surfaces (60) and a distally presented surface (62). In the current example, flat surfaces (60) form a general hexagonal shape. Flat surfaces (60) and distally presented surface (62) together define a recessed pocket (65) dimensioned to house second torque receiving body (14) of bone screwdriver bit (10). When second torque receiving body (14) is housed within recessed pocket (65), flat surfaces (60) are configured to transfer torque from handle body (52) onto torque receiving body (14).

Collar (64) defines a plurality of transverse locking pockets (66). As will be described in greater detail below, transverse locking pockets (66) house a respective locking body (90) of driver retention assembly (80); thereby allowing locking bodies (90) to actuate radially along a predetermined path relative to collar (64) between an unlocked configuration and a locked configuration. Once driver retention assembly (80) is assembled, locking bodies (90) are inhibited from disassociating with their respective locking pocket (66). Transverse locking pockets (66) are in communication with an external diameter and an internal diameter of collar (64) such that a portion of locking bodies (90) may operatively engage suitable portions of bone screwdriver bit (10) to thereby longitudinally retain bit (10) relative to handle (50) while driver retention assembly (80) is in the locked configuration in accordance with the teachings herein.

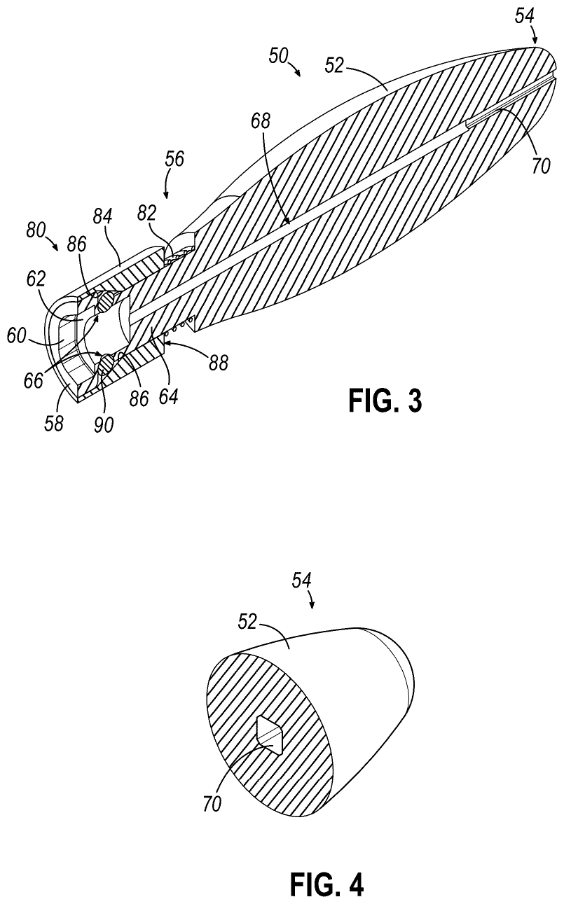

An internal diameter of collar (64) is in communication with recessed pocket (65). Turning to FIG. 3, the internal diameter of collar (64) and handle body (52) define an elongated shaft receiving channel (68). Shaft receiving channel (68) is dimensioned to receive and house elongated navigation unit coupling body (20) to thereby reduce the effective length of bone screwdriver bit (10) to the length (L3) of distal shaft (22) and screw bit (24).

As best shown in FIGS. 3-4, a proximal portion of shaft receiving channel (68) includes proximal torque transfer surfaces (70) that are dimensioned to suitably engage proximal torque receiving body (12) of bone screwdriver bit (10). When elongated navigation unit coupling body (20) is housed within shaft receiving channel (68), proximal torque transfer surfaces (70) are configured to transfer torque from handle body (52) onto torque receiving body (12). Therefore, while the effective length of bone screwdriver bit (10) is reduced by channel (68) housing elongated navigation unit coupling body (20), handle body (52) is still configured to transfer torque onto bone screwdriver bit (10) in order to suitably anchor pedicle screws (100) into the pedicle (P) of vertebra (V). While the current example includes distal torque transfer body (58) and proximal torque transfer surfaces (70), which are configured to transfer torque onto respective, suitable portions (e.g., 14 and 12) of bit (10); in some instances, only one transfer feature of handle (50) may be present.

As disclosed above, driver retention assembly (80) is configured to retain bone screwdriver bit (10) to inhibit inadvertent decoupling of bone screwdriver bit (10) from handle (50). In particular, driver retention assembly (80) is configured to actuate relative to handle body (52) between an unlocked configuration (which allows bit (10) to couple/decouple with handle (50)) and a locked configuration (which inhibits bit (10) from decoupling with handle (50) once suitably attached). As best shown in FIG. 2, driver retention assembly (80) includes a biasing element in the form of a spring (82), an actuating retention body in the form of a translating body (84), and a plurality of locking bodies (90).

In the current example, spring (82) is a coiled spring disposed around collar (64) of handle (50). Spring (82) is interposed between a distally presented surface of handle body (52) and a proximally presented surface of translating body (84). Spring (82) biases translating body (84) relative to handle (50) into the locked configuration (e.g., biases translating body (84) distally relative to handle (50)). As will be described in greater detail below, a user may proximally retract translating body (84) along collar (64) to thereby compress spring (82) in order to transition driver retention assembly (80) from the locked configuration into the unlocked configuration.

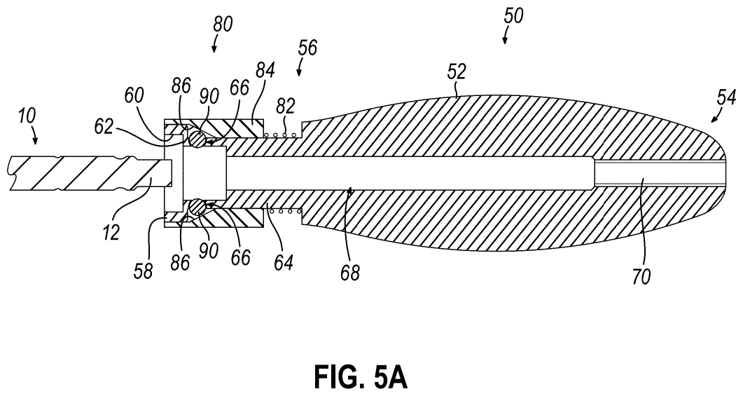

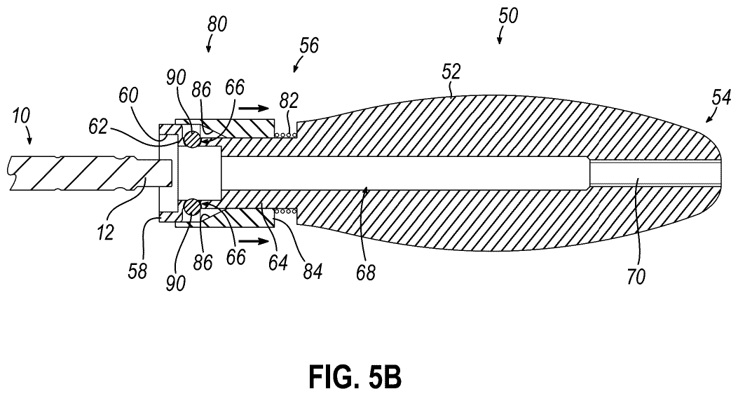

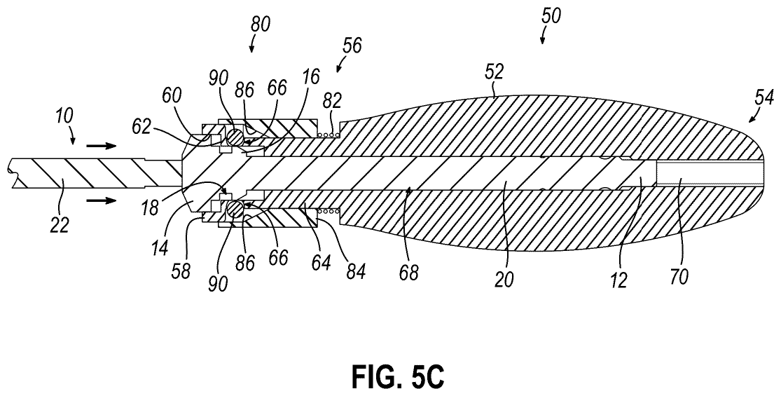

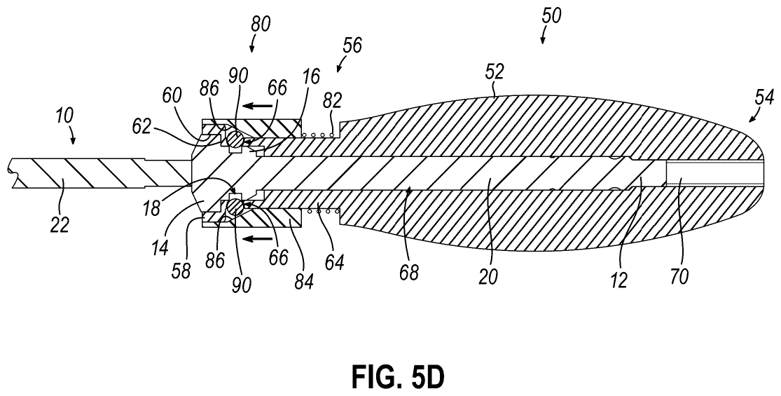

Translating body (84) defines a through hole (88) that is at least partially defined by a slanted locking surface (86). Translating body (84) is slidably disposed over collar (64) such that through hole (88) receives collar (64). As best shown in FIGS. 3, 5A, and 5D, slanted locking surface (86) is configured to engage locking bodies (90) in the locked configuration to thereby drive locking bodies (90) downward within locking pockets (66). In certain embodiments, translating body (84) is cylindrical in shape and includes an annular configuration as shown, for example, in FIG. 2. It is understood, however, that other shapes and configurations are possible for driver retention assembly (80) and its components.

Spring (82) biases translating body (84) distally such that slanted locking surface (86) urges locking bodies (90) into a respective locking pocket (66) (e.g., radially inward), thereby reaching the locked configuration (e.g., the natural state of spring (82)). While in the locked configuration, locking bodies (90) extend partially within shaft receiving channel (68) a suitable distance to engage suitable portions of bone screwdriver bit (10). Engagement between locking bodies (90) and bone screwdriver bit (10) in the locked configuration provides a frictional braking force onto bone screwdriver bit (10) to inhibit inadvertent decoupling of bit (10) from handle (50). Therefore, while locking bodies (90) are in the locked configuration and bit (10) is suitably coupled to handle (50), bit (10) may be inhibited from inadvertently decoupling with handle (50) during illustrative use.

A user may proximally retract translating body (84) such that slanted locking surface (86) no longer urges locking bodies (90) radially inward within their respective locking pocket (66), thereby reaching the unlocked configuration. While in the unlocked configuration, locking bodies (90) may be pushed radially away (e.g., radially outward) from their respective locking pocket (66) (see FIG. 5C) a suitable distance to no longer inhibit decoupling of bit (10) from handle (50). Therefore, while locking bodies (90) are in the unlocked configuration, bit (10) may be inserted and retracted from shaft receiving channel (68) such that any frictional braking force imparted on bit (10) via locking bodies (90) is negligible.

FIGS. 5A-5D show an illustrative coupling of bone screwdriver bit (10) with shaft reduction handle (50) in order to reduce the effective length of bit (10) to the length (L3) of distal shaft (22) and screw bit (24). As mentioned above, in some instances, it may be desirable to reduce the effective length of a navigation compatible bone screwdriver bit (10) when being used without the assistance of a navigation system.

As shown in FIG. 5A, a proximal end of bone screwdriver bit (10), including first torque receiving body (12), is aligned with distal end end (54) of shaft reduction handle (50). Therefore, first torque receiving body (12) is aligned to be inserted within shaft receiving channel (68) of handle body (52) at distal end (54).

As shown in FIG. 5B, a user actuates translating body (84) proximally such that spring (82) is compressed and such that slanted locking surface (86) is not urged against locking bodies (90); thereby actuating driver retention assembly (80) into the unlocked configuration. It should be understood that user may actuate driver retention assembly (80) into the unlocked configuration prior to suitably aligning bone screwdriver bit (10 with handle (50) or at any other suitable time as would be apparent to one skilled in the art in view of the teachings herein.

As shown in FIG. 5C, with driver retention assembly (80) held in the unlocked configuration, a user inserts proximal end of bone screwdriver bit (10), including elongated navigation unit coupling body (20), within shaft receiving channel (68). As also shown in FIG. 5C, with locking bodies (90) in the unlocked configuration, collar (16) of bone screwdriver bit (10) (or other suitable components of bit (10)) may urge locking bodies (90) radially outward from their respective locking pockets (66).

As shown in FIG. 5D, a user further inserts elongated navigation unit coupling body (20) into shaft receiving channel (68) of handle body (52) until second torque receiving body (14) it suitably housed within the recess defined by flat surfaces (60) and distally presented surface (62) of distal torque transfer body (58); and also until annular recess (18) defined by collar (16) and second torque receiving body (14) are longitudinally aligned with locking bodies (90).

It should be understood that first torque receiving body (12) is also suitably housed within proximal torque transfer body (70) of handle body (52). Therefore, when bone screwdriver bit (10) is inserted into the position shown in FIG. 5D, handle (50) is configured to suitably transfer torque onto bit (10) in order to anchor pedicle screws (100) in accordance with the teachings herein. Additionally, when bone screwdriver bit (10) is inserted into the position shown in FIG. 5D, handle (50) reduces the effective length of bit (10) such that the length (L2) of elongated navigation unit coupling body (20) is housed within the confines of handle body (52).

As also shown in FIG. 5D, a user releases translating body (84) such that spring (82) drives or biases slanted locking surface (86) into engagement with locking bodies (90). Locking bodies (90) are then driven radially inward into respective locking pockets (66) and annular recess (18); and into suitable engagement with collar (16) and/or second torque receiving body (14). As mentioned above, engagement between locking bodies (90) and suitable components of bone screwdriver bit (10) provides a frictional braking force that inhibits bone screwdriver bit (10) from inadvertently disassociating with handle (50) during illustrative use. With bit (10) suitably coupled to handle (50) at a reduced effective length, a user may utilize handle (50) and bit (10) in order to anchor pedicle screws (100) into the pedicle (P) of a patient's vertebra (V), as shown in FIG. 6. It should be understood if a user desired to decouple bit (10) from handle (50), the user may actuate translating body (84) back into the unlocked position and pull bit (10) out of handle (50) or pull handle (50) off of bit (10).

In the current example, locking bodies (90) are spherical. However, locking bodies (90) may include any suitable shape as would be apparent to one skilled in the art in view of the teachings herein. Additionally, in the current example, slanted locking surface (86) is annular with a relatively consistent tapered profile. However, slanted locking surface (86) may include any suitable shape as would be apparent to one skilled in are in view of the teachings herein.

In the current example, driver retention assembly (80) includes translating body (84) that actuates along the longitudinal axis in order to transition between the locked configuration and the unlocked configuration. However, any other suitable means may be used to transition between the locked and unlocked configuration as would be apparent to one skilled in the art in view of the teachings herein. For example, buttons that are pressed down in the radial direction may be used in order to transition between the locked and unlocked configuration.

In the current example, the driver retention assembly (80) is located at a distal end of handle body (52). However, driver retention assembly (80) may be located at any other suitable location as would be apparent to one skilled in the art. For example, driver retention assembly (80) may associate with distal torque transfer body (58), proximal torque transfer surface (70), or any other suitably location along handle body (52).

While proximal torque transfer surface (70) in the current example forms a square cross section, proximal torque transfer surfaces (70) may have any suitable geometry as would be apparent to one skilled in the art in view of the teachings herein. For example, proximal torque transfer surfaces may be 6-sided, oblong/elliptical, etc.

### II. Illustrative Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus comprising: (a) a handle extending between a proximal portion and a distal portion, the handle defining a shaft receiving channel extending from an end of the distal portion into the proximal portion of the handle, wherein the handle comprises at least one torque generating surface; and (b) a retention assembly associated with the handle, wherein the shaft receiving channel is dimensioned to receive an elongated navigation unit coupling body of a navigation compatible bone screwdriver bit to thereby reduce an effective length of the navigation compatible bone screwdriver bit, wherein the at least one torque generating surface is configured to transmit torque onto the navigation compatible bone screwdriver bit while the elongated navigation unit coupling body of the navigation compatible bone screwdriver bit is housed within the shaft receiving channel, wherein the retention assembly is configured to inhibit decoupling of the handle and the navigation compatible bone screwdriver bit while the elongated navigation unit coupling body of the navigation compatible bone screwdriver bit is housed within the shaft receiving channel.

### Example 2

The apparatus of Example 1, wherein the retention assembly is configured to transition between a locked configuration and an unlocked configuration, wherein retention assembly, while in the locked configuration, is configured inhibit decoupling of the handle and the navigation compatible bone screwdriver bit while the elongated navigation unit coupling body is housed within the shaft receiving channel, and wherein the retention assembly, while in the unlocked configuration, is configured permit decoupling of the handle and the navigation compatible bone screwdriver bit while the elongated navigation unit coupling body is housed within the shaft receiving channel.

### Example 3

The apparatus of Example 2, wherein the retention assembly comprises a bias spring configured to bias the retention assembly toward the locked configuration.

### Example 4

The apparatus of Example 3, wherein the bias spring comprises a coil spring.

### Example 5

The apparatus of Example 4, wherein handle comprises a collar, wherein the coil spring is disposed around the collar.

### Example 6

The apparatus of any one or more of Examples 1 through 5, wherein the retention assembly comprises a locking body configured to, while the retention assembly is in the locked configuration, impart a frictional braking force on the navigation compatible bone screwdriver bit while the elongated navigation unit couple boys is housed within the shaft receiving channel.

### Example 7

The apparatus of Example 6, wherein the handle further defines a transverse locking pocket in communication with the shaft receiving channel, wherein the locking body is at least partially housed within the transverse locking pocket.

### Example 8

The apparatus of Example 7, wherein the locking body comprises a sphere.

### Example 9

The apparatus of either Example 7 or 8, wherein the retention assembly further comprises a translating body interposed between the bias spring and the locking body.

### Example 10

The apparatus of any one or more of Examples 5 through 9, wherein the translating body comprises a slanted surface configured to engage the locking body while the retention assembly is in the locked configuration.

### Example 11

The apparatus of Example 10, wherein the translating body is slidably disposed on the handle.

### Example 12

The apparatus of any one or more of Examples 1 through 11, wherein the at least one torque generating surface comprising a first torque generating surface defining a portion of the shaft receiving channel.

### Example 13

The apparatus of Example 12, wherein the first torque generating surface is located at the proximal portion of the handle.

### Example 14

The apparatus of Example 13, wherein the at least one torque generating surface comprises a second torque generating surface located distally relative to the retention assembly.

### Example 15

The apparatus of Example 14, wherein the second torque generating surface forms at least a portion of a hexagonal shape.

### Example 16

An apparatus comprising: (a) a handle extending between a proximal portion and a distal portion, the handle defining a shaft receiving channel, wherein the handle comprises at least one torque generating surface defining at least a portion of the shaft receiving channel; and (b) a retention assembly associated with the handle, wherein the retention assembly comprises: (i) a locking body configured to move relative to the handle between a locked configuration and an unlocked configuration, and (ii) bias spring configured to bias the locking body toward the locked configuration; wherein the shaft receiving channel is dimensioned to receive an elongated navigation unit coupling body of a navigation compatible bone screwdriver bit to thereby reduce an effective length of the navigation compatible bone screwdriver bit, wherein the at least one torque generating surface is configured to transmit torque onto the navigation compatible bone screwdriver bit while the elongated navigation unit coupling body of the navigation compatible bone screwdriver bit is housed within the shaft receiving channel, wherein the retention assembly, while in the locked configuration, is configured to inhibit decoupling of the handle and the navigation compatible bone screwdriver bit while the elongated navigation unit coupling body of the navigation compatible bone screwdriver bit is housed within the shaft receiving channel.

### Example 17

The apparatus of Example 16, wherein the retention assembly further comprises a cam surface interposed between the bias spring and the locking body.

### Example 18

The apparatus of Example 17, wherein the retention assembly comprises a translating body associated with the cam surface.

### Example 19

The apparatus of Example 18, wherein the cam surface comprises a slanted locking surface.

### Example 20

An apparatus comprising: (a) a handle extending between a proximal portion and a distal portion, the handle defining a shaft receiving channel extending from an end of the distal portion into the proximal portion of the handle, wherein the handle comprises at least one torque generating surface; and (b) a retention assembly coupled with the distal portion of the handle, wherein the retention assembly comprises a locking body, wherein the shaft receiving channel is dimensioned to receive an elongated navigation unit coupling body of a navigation compatible bone screwdriver bit to thereby reduce an effective length of the navigation compatible bone screwdriver bit, wherein the at least one torque generating surface is configured to transmit torque onto the navigation compatible bone screwdriver bit while the elongated navigation unit coupling body of the navigation compatible bone screwdriver bit is housed within the shaft receiving channel, wherein the locking body of the retention assembly is configured to impart a frictional braking force on the navigation compatible bone screwdriver bit to thereby inhibit decoupling of the handle and the navigation compatible bone screwdriver bit while the elongated navigation unit coupling body of the navigation compatible bone screwdriver bit is housed within the shaft receiving channel.

### III. Miscellaneous

It should also be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) a handle extending between a proximal portion and a distal portion, the handle defining a shaft receiving channel extending from an end of the distal portion into the proximal portion of the handle, wherein the handle comprises at least one torque generating surface; and
(b) a retention assembly associated with the handle, wherein the shaft receiving channel is dimensioned to receive an elongated navigation unit coupling body of a navigation compatible bone screwdriver bit to thereby reduce an effective length of the navigation compatible bone screwdriver bit,
wherein the at least one torque generating surface is configured to transmit torque onto the navigation compatible bone screwdriver bit while the elongated navigation unit coupling body of the navigation compatible bone screwdriver bit is housed within the shaft receiving channel,
wherein the retention assembly is configured to inhibit decoupling of the handle and the navigation compatible bone screwdriver bit while the elongated navigation unit coupling body of the navigation compatible bone screwdriver bit is housed within the shaft receiving channel.

2. The apparatus of claim 1, wherein the retention assembly is configured to transition between a locked configuration and an unlocked configuration,
wherein retention assembly, while in the locked configuration, is configured inhibit decoupling of the handle and the navigation compatible bone screwdriver bit while the elongated navigation unit coupling body is housed within the shaft receiving channel, and
wherein the retention assembly, while in the unlocked configuration, is configured permit decoupling of the handle and the navigation compatible bone screwdriver bit while the elongated navigation unit coupling body is housed within the shaft receiving channel.

3. The apparatus of claim 2, wherein the retention assembly comprises a bias spring configured to bias the retention assembly toward the locked configuration.

4. The apparatus of claim 3, wherein the bias spring comprises a coil spring.

5. The apparatus of claim 4, wherein handle comprises a collar, wherein the coil spring is disposed around the collar.

6. The apparatus of claim 3, wherein the retention assembly comprises a locking body configured to, while the retention assembly is in the locked configuration, impart a frictional braking force on the navigation compatible bone screwdriver bit while the elongated navigation unit couple boys is housed within the shaft receiving channel.

7. The apparatus of claim 6, wherein the handle further defines a transverse locking pocket in communication with the shaft receiving channel, wherein the locking body is at least partially housed within the transverse locking pocket.

8. The apparatus of claim 7, wherein the locking body comprises a sphere.

9. The apparatus of claim 7, wherein the retention assembly further comprises a translating body interposed between the bias spring and the locking body.

10. The apparatus of claim 5, wherein the translating body comprises a slanted surface configured to engage the locking body while the retention assembly is in the locked configuration.

11. The apparatus of claim 10, wherein the translating body is slidably disposed on the handle.

12. The apparatus of claim 1, wherein the at least one torque generating surface comprising a first torque generating surface defining a portion of the shaft receiving channel.

13. The apparatus of claim 12, wherein the first torque generating surface is located at the proximal portion of the handle.

14. The apparatus of claim 13, wherein the at least one torque generating surface comprises a second torque generating surface located distally relative to the retention assembly.

15. The apparatus of claim 14, wherein the second torque generating surface forms at least a portion of a hexagonal shape.

16. An apparatus comprising:
(a) a handle extending between a proximal portion and a distal portion, the handle defining a shaft receiving channel, wherein the handle comprises at least one torque generating surface defining at least a portion of the shaft receiving channel; and
(b) a retention assembly associated with the handle, wherein the retention assembly comprises:
(i) a locking body configured to move relative to the handle between a locked configuration and an unlocked configuration, and
(ii) bias spring configured to bias the locking body toward the locked configuration;
wherein the shaft receiving channel is dimensioned to receive an elongated navigation unit coupling body of a navigation compatible bone screwdriver bit to thereby reduce an effective length of the navigation compatible bone screwdriver bit,
wherein the at least one torque generating surface is configured to transmit torque onto the navigation compatible bone screwdriver bit while the elongated navigation unit coupling body of the navigation compatible bone screwdriver bit is housed within the shaft receiving channel,
wherein the retention assembly, while in the locked configuration, is configured to inhibit decoupling of the handle and the navigation compatible bone screwdriver bit while the elongated navigation unit coupling body of the navigation compatible bone screwdriver bit is housed within the shaft receiving channel.

17. The apparatus of claim 16, wherein the retention assembly further comprises a cam surface interposed between the bias spring and the locking body.

18. The apparatus of claim 17, wherein the retention assembly comprises a translating body associated with the cam surface.

19. The apparatus of claim 18, wherein the cam surface comprises a slanted locking surface.

20. An apparatus comprising:
(a) a handle extending between a proximal portion and a distal portion, the handle defining a shaft receiving channel extending from an end of the distal portion into the proximal portion of the handle, wherein the handle comprises at least one torque generating surface; and
(b) a retention assembly coupled with the distal portion of the handle, wherein the retention assembly comprises a locking body,
wherein the shaft receiving channel is dimensioned to receive an elongated navigation unit coupling body of a navigation compatible bone screwdriver bit to thereby reduce an effective length of the navigation compatible bone screwdriver bit,
wherein the at least one torque generating surface is configured to transmit torque onto the navigation compatible bone screwdriver bit while the elongated navigation unit coupling body of the navigation compatible bone screwdriver bit is housed within the shaft receiving channel,
wherein the locking body of the retention assembly is configured to impart a frictional braking force on the navigation compatible bone screwdriver bit to thereby inhibit decoupling of the handle and the navigation compatible bone screwdriver bit while the elongated navigation unit coupling body of the navigation compatible bone screwdriver bit is housed within the shaft receiving channel.
